(19) United States
(12) Patent Application Publication
(10) Pub. No.: US 2014/0213933 A1
(43) Pub. Date: Jul. 31, 2014

(54) DEVICE FOR THE LAPAROSCOPIC REMOVAL OF A SUPERFICIAL CYLINDRICAL SAMPLE OF A HUMAN OR ANIMAL BODY TISSUE

(71) Applicants: Cecile Real, Paris (FR); Patrick Henri, Bois Colombes (FR)

(72) Inventors: Cecile Real, Paris (FR); Patrick Henri, Bois Colombes (FR)

(21) Appl. No.: 14/343,049

(22) PCT Filed: Sep. 5, 2012
(86) PCT No.: PCT/EP2012/067268

§ 371 Date: Mar. 5, 2014

(30) Foreign Application Priority Data
Sep. 5, 2011 (FR) 1157854

(51) Int. Cl. A61B 10/04; A61B 10/02

(52) U.S. Cl. CPC A61B 10/04; A61B 10/0266; A61B 2010/045
USPC 600/567

(57) ABSTRACT

The invention concerns a device for the laparoscopic removal of a superficial cylindrical sample of a human or animal body tissue, comprising:—a tube (1) whose inner diameter determines the diameter of the sample to be removed, and having a cutting edge (12) at its distal end (10),—a grip handle (2), fixed to the tube (1) in the region of a proximal end (11) of said tube(1),—a knife (3) arranged inside the tube (1) and having a cutting edge (31),—a device to actuate the knife (3), comprising an actuating element (22, 41) connected to the grip handle (2) in such a way as to give the knife (3) a helical movement in the direction of a distal end of the tube (1), followed by a rotational movement to detach the sample from the tissue.

### FIELD OF THE INVENTION

The present invention relates to a device for laparoscopically collecting a cylindrical surface sample of a human or animal body tissue.

### BACKGROUND OF THE INVENTION

Biopsy is a procedure widely used for diagnostic purposes.

In fact, a sample collected from a tissue of the human or an animal body can be analyzed in a laboratory for the purpose of detecting possible lesions, diseases, etc.

In order to limit post-operative effects on the patient, laparoscopic procedures wherein a collecting instrument is inserted into the body through a small incision, have been developed.

Among the tissues wherein a biopsy may be performed are distinguished in particular the solid organs, which can be considered as volumes, and the membranes, wherein the surface area is great compared to the thickness.

Thus for example, biopsy for diagnosis of endometriosis is carried out in the peritoneum, which is a relatively thin membrane (a few millimeters) surrounding the viscera.

In such a case, the aim is to collect a surface sample of the peritoneum without perforating it, which implies that the thickness of the sample is less than the thickness of the peritoneum.

Known biopsy needles, however, make it possible to collect, in a solid organ such as the liver for example, wherein they penetrate deeply, a thick core having only a small diameter (for example, a cylinder on the order of 1 mm in diameter by 5 to 6 mm thick).

Such needles are not suited for sampling on the peritoneum because they involve perforation thereof, resulting in a considerable trauma to the patient.

A need therefore exists for a device for laparoscopically collecting a sample of an internal human or animal body tissue when said tissue is a body membrane or a thin organ.

One object of the invention is therefore to obtain a device usable in laparoscopy to collect a surface sample of a tissue, of a membrane for example, without perforating said tissue.

### BRIEF DESCRIPTION OF THE INVENTION

In conformity with the invention, a device is proposed for laparoscopically collecting a surface sample of a human or animal body tissue, characterized in that it includes:
a tube the inner diameter whereof determines the diameter of the sample to be collected, and having a cutting edge at its distal end;
a gripping handle, fastened to the tube in the region of a proximal end of said tube;
a knife positioned inside the tube and having a cutting edge;
a device for actuating the knife including an actuating element connected to the gripping handle so as to give the knife a helical motion in the direction of a distal end of the tube, then a rotary motion to detach the sample from the tissue.

With particular advantage, the actuating element is also arranged so that, after said rotary motion and under the influence of pressure exerted in the direction of the distal end, it causes the knife to slide inside the tube so as to cause it to protrude from the distal end of the tube for the purpose of releasing the collected sample.

According to one advantageous embodiment of the invention, said actuating device includes:
a connecting element fastened to the tube,
an actuating rod fastened, at its distal end, to the knife,
a retaining element fastened to the actuating rod, connected to the connecting element by a helical connection in a proximal position of the actuating rod relative to the tube and by a pivoting connection in a distal position of the actuating rod.

According to one particular embodiment of the invention, said connecting element includes a tapped proximal portion cooperating with a threaded collar of the retaining element to form said helical connection and a distal bore larger than said collar to form said pivoting connection.

Moreover, said actuating device can include a thumbwheel connected to the distal end of the actuating rod.

According to a particular embodiment of the invention, the retaining element is held in the connecting element by a ring, said ring including a proximal stop for the retaining element with respect to the connecting element, said stop being retractable to release the retaining element from the connecting element.

For example, the inner diameter of the tube is comprised between 2 and 5 mm.

As for the longitudinal travel of the knife in its helical motion, it can be comprised between 1 and 5 mm.

According to a preferred embodiment of the invention, the gripping handle has a generally pistol-like shape, the actuating element including a trigger.

Said handle also advantageously includes a safety button movable between a locking position and a position releasing the trigger.

### BRIEF DESCRIPTION OF THE DRAWINGS

Other features and advantages of the invention will appear from the detailed description that follows, with reference to the appended drawings wherein:

FIG. 1 shows side and section views of a preferred embodiment of the actuating device according to the invention, in an initial collecting position,

FIG. 2 shows side, front, top and section views of a preferred embodiment of the invention,

FIG. 3 shows side and section views of a second embodiment of the invention,

FIGS. 4A through 4E illustrate different steps in using the device to perform a sample collection.

### DETAILED DESCRIPTION OF THE INVENTION

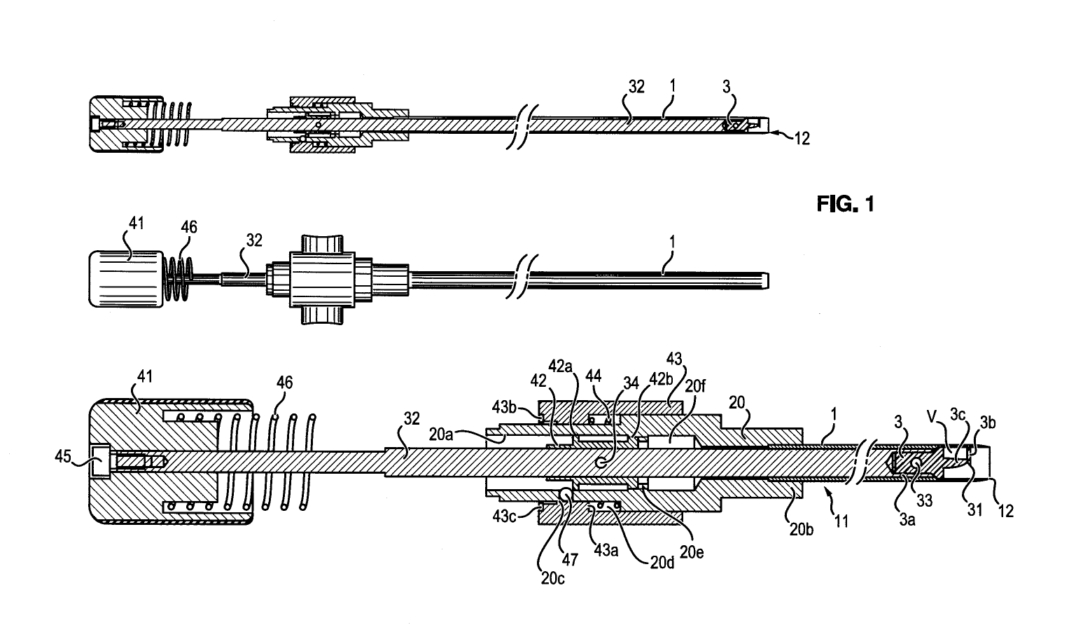

FIG. 1 shows a side view and a section view of the actuating device, with details A and B of the distal portion and of the proximal portion of the device, respectively.

In the present text, the side closest to the tissue wherein the sample must be collected is called “distal,” and the opposite side, closest to the user's hand, is called “proximal.”

The tube 1 is a tube the inner diameter whereof is equal to the diameter of the sample to be collected.

For example, the inner diameter of the tube 1 can be comprised between 2 and 5 mm.

The length of the tube can depend on the location of the tissue to be collected in the human or animal body.

Typically, the length of the tube 1 can be on the order of 300 mm.

The distal end 10 of the tube 1 has a cutting edge 12, similar to that of a hole punch.

The tube 1 is thus suited for penetrating into the tissue while cutting the peripheral wall of the sample under the influence of pressure exerted by a user.

Within the tube 1 is positioned a knife 3 which, when it is in a suitable position relative to the tube 1, allows the bottom of the sample to be detached, that is the disk located within the depth of the tissue.

To this end, the knife 3 has a cutting edge 31.

Preferably, the cutting edge 31 is perpendicular to the tube wall 1, so that a rotary motion of the knife 3 makes it possible to cut the sample in a direction perpendicular to its cylindrical wall.

The knife 3 advantageously exhibits a generally cylindrical proximal portion 3a designed to be fastened to an actuating rod 32 (described below) and a circular arc distal portion 3b bearing the cutting edge 31.

The cutting edge 31 is preferably rectilinear along a diameter of the circular arc distal portion 3b. Alternatively, the cutting edge can be curved or have any other appropriate profile.

The portions 3a and 3b have an outer diameter substantially equal to the inner diameter of the tube 1.

Said portions 3a and 3b are connected by a leg 3c the diameter whereof is less than the inner diameter of the tube 1.

As will be seen below, the volume V comprised between the inner wall of the tube 1, the distal face of the portion 3a and the proximal face of the portion 3b of the knife defines the volume of the sample to be collected.

The knife 3 is fastened to the actuating rod 32 by its proximal portion 3a.

For example, as illustrated in FIG. 1, the proximal portion 3a of the knife is partially inserted into the distal end of the rod 32 and fastened within it by means of a pin 33. Naturally, any other means of attachment can be used.

The actuating rod 32 has a diameter slightly smaller than the inner diameter of the tube 1 so as to be able to slide in translation and in rotation within it without friction and to have mechanical resistance suited to the use.

At its proximal end, the actuating rod 32 is fastened to a thumbwheel 41.

In the example illustrated here, the thumbwheel 41 is fastened on the operating rod 32 by means of a screw 45, but it is self-evident that any other fastening means could be employed without thereby departing from the scope of the present invention.

Moreover, the actuating rod 32 also bears, in its proximal portion, a retaining element 42.

Said retaining element 42 has a generally cylindrical shape, with an inner diameter slightly greater than that of the actuating rod 32 so as to be able to be slipped over it.

In the example illustrated here, the retaining element 42 is fastened to the actuating rod 32 by means of a pin 34, but the invention is of course not limited to this fastening method.

In its proximal portion, the retaining element 42 has a smooth collar 42a and, in its distal portion, a threaded collar 42b.

Moreover, the sample collecting device includes a gripping handle 2.

The handle 2 includes a connecting element 20 which makes it possible to connect, through the retaining element 42, the actuating rod 32 (which as mentioned above is fastened to the thumbwheel 41 and the knife 3) with the tube 1.

More precisely, said connecting element 20 exhibits a generally cylindrical shape with an internal passage having an inner diameter greater than the outer diameter of the actuating rod 32 to allow insertion and sliding of said rod 32 in said passage.

From its proximal portion toward its distal portion, the internal passage includes in succession a recess 20a, the inner diameter whereof is greater than the outer diameter of the collars 42a and 42b, a tapped bore 20e designed to cooperate with the thread of the collar 42b, a recess 20f, the inner diameter whereof is greater than the diameter of the collar 42b.

In its distal portion 20b, the connecting element 20 is fastened to the proximal end 11 of the tube 1.

Moreover, the connecting element 20 is surrounded by a ring 43.

Said ring 43 is arranged so as to slide over an outer wall of the connecting element 20 in the direction of the distal end of the device, against the reaction of an elastic element 44, which is for example a tension spring.

Said spring 44 is housed between an inner shoulder 20d of the connecting element 20 and an inner shoulder 43a of the ring 43.

In its proximal portion, the ring 43 is held in abutment with respect to the connecting element 20 by a clip 43b.

In its portion that is surrounded by the ring 43, the passage 20a of the connecting element 20 includes a through opening 20c constituting an accommodation for a ball 47.

The ball 47 has a diameter greater than the depth of the opening 20c within which it is accommodated, so that in a first position of the device, called the initial sample collection position, the ball 47 protrudes toward the inside of the passage 20a with respect to the wall of said passage.

In this position, the threaded collar 42b of the retaining element 42 described above is screwed into a tapped bore 20e of the connecting element 20.

The tapped bore 20e has a length greater than the length of the threaded collar 42b.

The proximal collar 42a of the retaining element 42 is situated in the distal portion of the passage 20a with respect to the ball 47, so that the ball 47 constitutes a stop in the proximal direction for the actuating rod 32.

When the ring 43 is driven in the distal direction against the spring 44, a cavity 43c formed in the inner wall of the ring 43 comes face-to-face with the through opening 20c containing the ball 47. The cavity 43c has sufficient depth for the ball 47 to then be completely retracted into the through opening 20c and the cavity 43c and therefore does not impede sliding of the stop and of the actuating rod 32 in the proximal direction.

Thus, by pressing on the ring 43 in the distal direction, the collecting device can be separated into two subassemblies: the actuating rod 32, the knife 3 and the thumbwheel 41 on the one hand, and the connecting element 20, the tube 1 and the ring 43 on the other.

As will be shown below, this ability of the device to be disassembled is advantageous because it allows better cleaning and sterilization of the device, which makes it suitable for multiple use.

To this end, the components of the device mentioned above are made of materials compatible with sterilization.

Among the preferred materials may be mentioned: stainless steel, polyether etherketone (PEEK), polyphenylsulfone (Radel®), but a specialist can choose any other material satisfying the required mechanical and sterilizability criteria.

However, the device can also be designed for single use and the disassembly, reassembly and sterilization constraints need not then be taken into account in designing said device.

Consequently, while maintaining the sample collection method described above (that is with a helical motion followed by a rotary motion of the knife), the design of the device and the materials employed may differ from those of the examples describe in order to obtain a single-use device that is optimal from the standpoint of cost, conditions of use, etc.

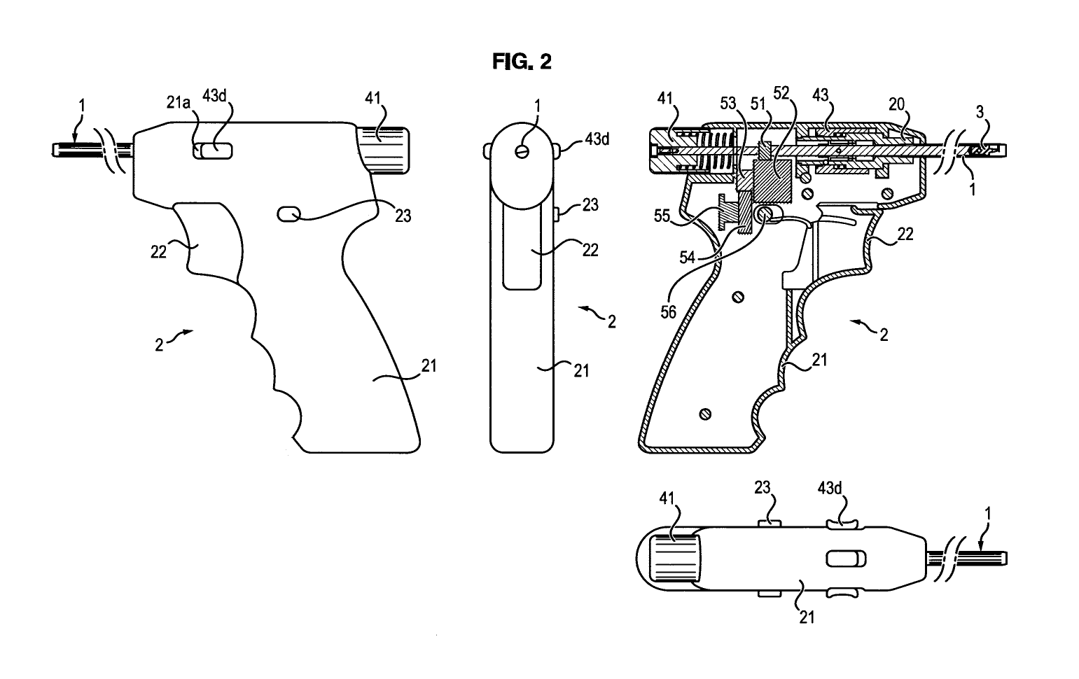

According to a first embodiment of the invention, illustrated in FIG. 2, the gripping handle 2 takes the form of a pistol equipped with a trigger for actuating the knife.

This design of the device is particularly advantageous because the handle has an ergonomic shape similar to that of other surgical tools, so that the user will use this new device intuitively.

This particular form is naturally not limiting, and any other ergonomic shape can be defined for the handle without thereby departing from the scope of the present invention.

The handle includes a case 21 having the general shape of a pistol including a gripping area designed for gripping by a user's hand.

The case can be made from two substantially symmetrical shells which can be assembled in a manner allowing disassembly (by means of screws for example) to allow access to the actuating device for the purpose of repair, as necessary.

The actuating device described above is assembled inside said case.

The connecting element 20 is preferably held in an appropriate accommodation inside the case, two passage openings being provided on the case for the tube 1, on the one hand, and for the thumbwheel 41 on the other.

Moreover, an elastic element 46, a tension spring for example, is interposed between the case and the thumbwheel 41 so as to exert a reaction in the distal direction on the thumbwheel.

Moreover, the ring 43 is provided on at least one of its sides with an operating button 43d. Said button 43d protrudes with respect to the outer wall of the case 21 through an oblong hole 21a. This allows the user to operate the ring 43 even though it is enclosed inside the case 21.

The handle also includes a trigger 22 braced against an elastic element 56, which is connected to the actuating rod by a toothed pulley 55 and a series of gears including toothed wheels 51, 52, 53 and 54.

In particular, the toothed wheel 51 is fastened to the actuating rod 32 so that pressure exerted on the trigger is transformed into a rotary motion of the actuating rod.

The travel of the trigger 22 when pulled is defined so that a first part of the travel corresponds to screwing the collar 42b into the bore 20e and a second portion of the travel corresponds to rotating the collar 42b into the accommodation 20f.

Thus, a single pull on the trigger 22 allows a tissue sample to be detached.

Advantageously, the handle also includes a safety button 23 which is movable between a position wherein it blocks the operation of the trigger (which avoids any unintentional operation) and a position wherein it releases it.

To operate the trigger, the user must therefore simultaneously press said safety button 23.

Once the trigger is pulled, it remains in that position and pressure exerted on the safety button 23, combined with the return force of the elastic element 56, allows it to be returned to its initial position.

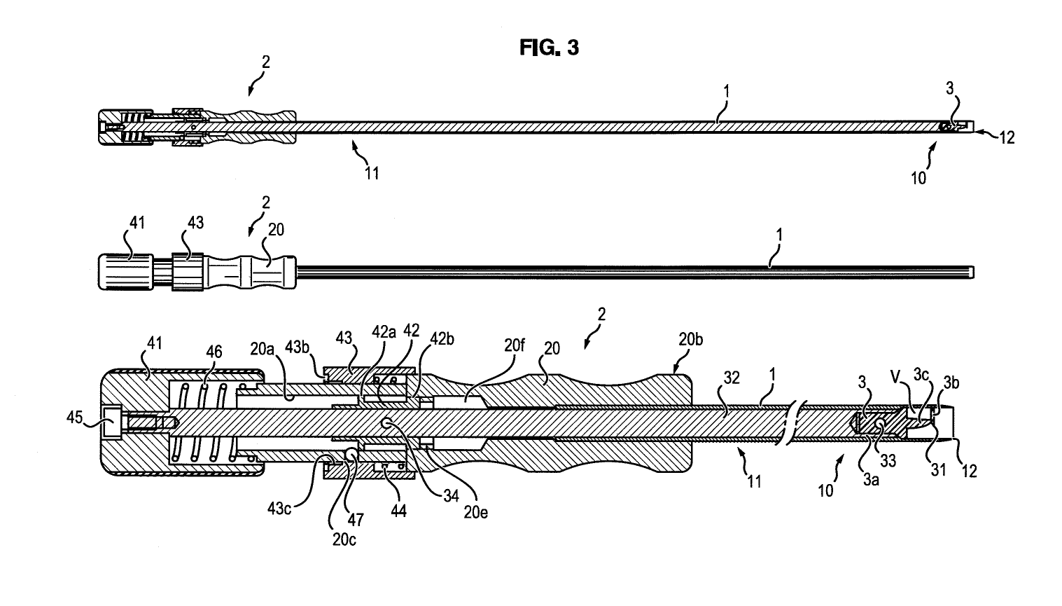

According to a second embodiment of the device, illustrated in FIG. 3, the gripping handle 2 has a tubular shape including a first portion fastened to the tube 1 and a second portion arranged for rotation and sliding with respect to the first portion and fastened to the actuating rod 32.

Said first portion includes the connecting element 20 described above which therefore, in this embodiment, fulfils both the function of connecting the tube 1 and the actuating rod 32 and the function of gripping by a user.

To this end, the outer wall of the connecting element 32 is advantageously provided with shapes suited to handling by a user.

Said second portion includes the subassembly consisting of the thumbwheel 41, the actuating rod 32 and the knife 3, described above.

In this embodiment, an elastic element 46 (a tension spring for example) is interposed between the thumbwheel 41 and the connecting element 20.

In the initial sample collection position mentioned above, the elastic element 46 is in the free or only slightly restrained state.

The retaining element 42 being in proximal abutment against the ball 47, rotation of the thumbwheel 41 in the counter-clockwise direction is impossible.

It is possible, however, to turn the thumbwheel 41 clockwise.

As long as the threaded collar 42b of the retaining element 42 is in engagement with the tapped bore 20e of the accommodation 20, the movement of the actuating rod 32 (and hence of the knife 3) is a helical motion in the distal direction.

Once the threaded collar 42b of the retaining element 42 is no longer in engagement with the tapped bore 20e of the connecting element 20, said threaded collar 42b is situated in the accommodation 20f.

In this position, called the sample detachment position, clockwise rotation of the thumbwheel 41 leads to pure rotation of the actuating rod 32 (and hence of the knife 3), that is to say a rotation without displacement in the distal direction.

Finally, starting from this detachment position, it is also possible to exert pressure on the thumbwheel 41 in the distal direction against the elastic element 46.

The length of the accommodation 20f then defines a travel corresponding to the travel by which the knife 3 is withdrawn from the tube 1.

This position, wherein the knife protrudes from the distal end 10 of the tube 1 is called the sample release position.

The procedure for using the device is the following.

In the figures that show this procedure, it is the first embodiment of the device (that is to say that in which the gripping handle is the pistol grip described above and illustrated in FIG. 2) which is shown.

However, unless the contrary is stated, the procedure is identical for the other embodiments of the device.

Before proceeding with sample collection, the user checks that the device is in the initial sample collection position, that is that the knife 3 (through the actuating rod 32 and the retaining element 42) is in proximal abutment in the tube 1.

To this end, the user tries to turn the thumbwheel 41 counter-clockwise.

If rotation is impossible, the device is in the initial position desired.

Otherwise, the user turns the thumbwheel 41 counter-clockwise until it comes into abutment.

The cutting out of the sample is carried out in two steps: a first step consisting of cutting the cylindrical wall of the sample, followed by a step consisting of detaching the tissue sample from the tissue by cutting away the face of the sample located in depth in the tissue.

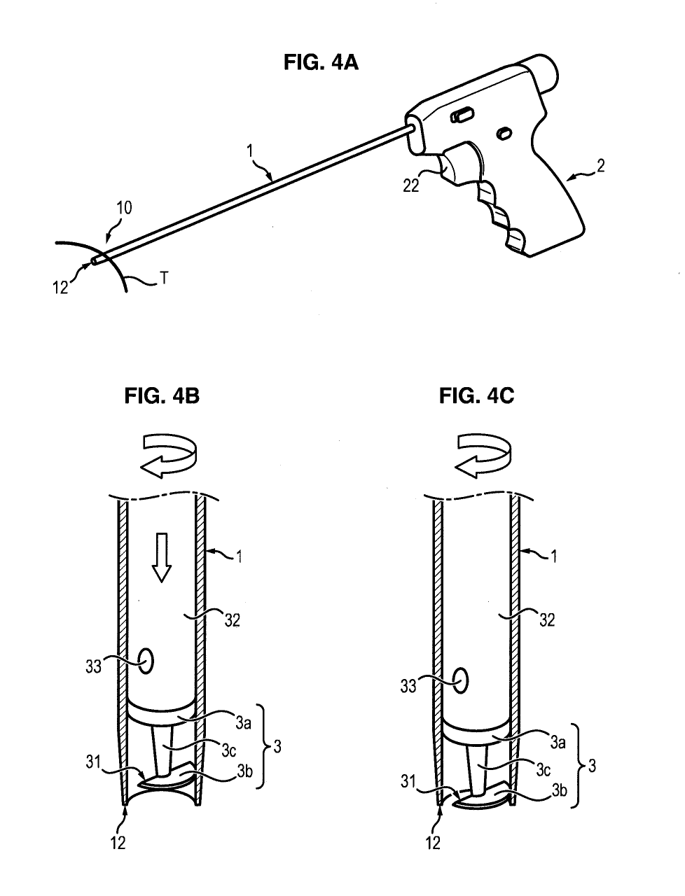

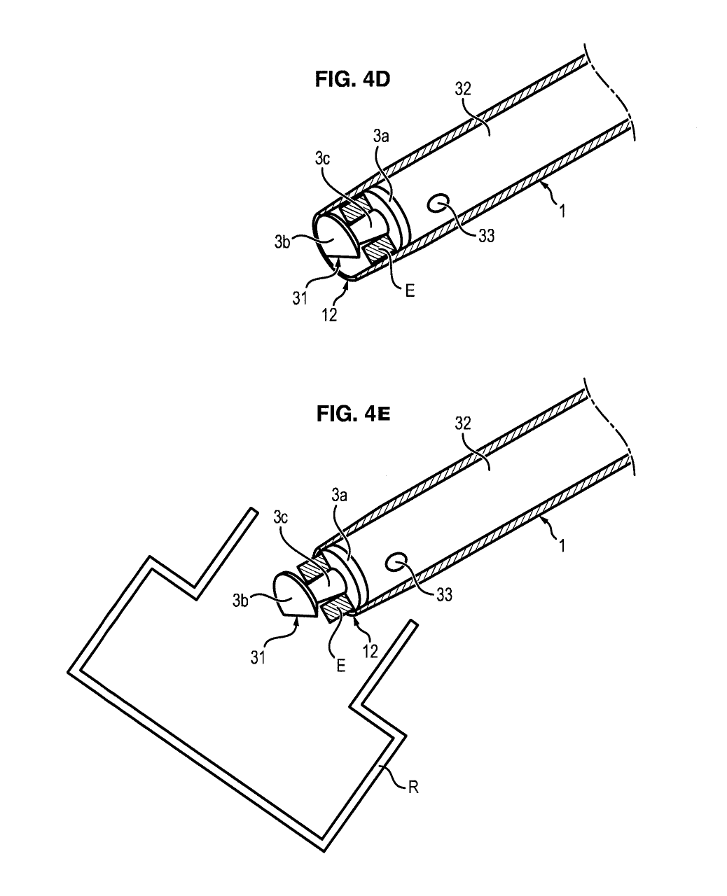

In the first cutting out step, illustrated in FIG. 4A, the user inserts the tube 1 through a cannula (not shown here) and drives the distal portion 10 of the tube 1 into the tissue T wherein he wishes to collect the sample.

The tube 1 fulfils the function of a hole punch by cutting out the cylindrical wall by means of its cutting edge 12.

The depth to which the tube 1 is driven depends on the nature of the tissue wherein the sample is collected; it is typically comprised between 1 and 5 mm.

Once the desired depth is reached, the user operates the actuating device for the purpose of detaching the sample.

In the case where the gripping handle is a pistol grip as illustrated in FIG. 2, the user pulls the trigger 22.

In the case where the gripping handle is a cylindrical handle as illustrated in FIG. 3, the user holds the connecting element 20 in the palm of his hand and turns the thumbwheel 41 clockwise while holding it between the thumb and the index finger.

Naturally, any other form of gripping handle can be defined, and any other appropriate means to this end can be designed to implement the actuating device without thereby departing from the scope of the present invention.

In any case, this action by the user has the effect of advancing the knife 3 within the tube 1 with a helical motion (FIG. 4B). This corresponds to screwing the threaded collar 42b of the retaining element 42 into the tapped bore 20e of the connecting element 20.

After a few turns (two or three for example), the advancement of the knife 3 is stopped because the threaded collar 42b is no longer in engagement with the tapped bore 20e and is located within the accommodation 20f.

When the user continues to turn the thumbwheel 41, the knife 3 is then given a purely rotary motion which allows the bottom of the sample to be cut away (FIG. 4C).

As can be seen in FIG. 4D, the sample E is then trapped in the tube 1 and held inside the tube 1 by the knife 3, which makes it possible to ensure that the sample does not fall out of the device when it is withdrawn from the patient's body.

The user withdraws the device from the cannula and inserts the distal end of the tube 1 into a container R designed for transporting the sample to the analysis site.

He then exerts pressure on the thumbwheel 41 to withdraw the knife 3 from the tube 1 (FIG. 4E).

The sample is released and falls into the container, by gravity and/or with the help of an appropriate tool (needle, forceps, etc.).

If necessary, the user can carry out one or several more sample collections in the same tissue.

To this end, he must again put the device into the initial position mentioned above and repeat the steps described above.

Starting from the sample release position, this involves releasing the pressure exerted on the thumbwheel 41 which, under the influence of the reaction of the elastic element 46, resumes its free proximal position.

In the embodiment wherein the gripping handle is the pistol grip of FIG. 2, the user must also press on the safety button, which brings the trigger 22 back into a protruding position and the retaining element 42 into abutment against the ball 47.

In the embodiment wherein the gripping handle is the cylindrical handle of FIG. 3, the user then turns the thumbwheel 41 counterclockwise, which results in screwing the collar 42b of the retaining element 42 into the tapped bore 20e of the connecting element 20, in the proximal direction. At the same time, the knife 3 enters the tube 1. When the proximal collar 42a of the retaining element 42 comes into abutment against the ball 47, the user can no longer turn the thumbwheel counter-clockwise and knows that the device is in its initial sample collection position.

After use, the device can be cleaned and sterilized if it is designed to be reusable, or discarded if it is single-use.

If necessary, to facilitate cleaning, the device is disassembled into the two subassemblies mentioned above.

The procedure for disassembling a reusable device is the following.

The user moves the ring 43 in the distal direction (if necessary, using the operating button 43d) which leads to retraction of the ball 47 into the through opening 20c and the cavity 43c.

The user then turns the thumbwheel 41 counter-clockwise to unscrew the collar 42b of the retaining element 42 from the tapped bore 20e of the connecting element 20.

Once the collar 42b is no longer in engagement with the bore 20e, the user can then withdraw from the connecting element 20 the subassembly consisting of the thumbwheel 41, the actuating rod 32, the retaining element 42 and the knife 43.

The other subassembly consists of the connecting element 20, the tube 1 and the ring 43.

Preferably, the elastic element 46 is fastened to the connecting element 20 or the thumbwheel 41, which avoids any risk of loss and facilitates the handling of dispersed parts.

The two subassemblies can then be cleaned and sterilized.

The procedure for reassembling the device is the following.

The user grasps the connecting element 20 and moves the ring 43 in the distal direction.

He can then insert the subassembly consisting of the thumbwheel 41, the actuating rod 32, the retaining element 42 and the knife 43 into the connecting element 20.

He turns the thumbwheel 41 clockwise to screw the collar 42b of the retaining element 42 into the tapped bore 20e of the connecting element.

He then releases the ring 43 which, under the reaction of the elastic element 44, resumes its free proximal against the clip 43b. At the same time, the ball 47 is forced out of the cavity 43c and protrudes inward again with respect to the wall of the passage 20a.

It is self-evident that the helical, then rotary motion of the knife can be provided by other elements without thereby departing from the scope of the present invention.

1. A device for laparoscopically collecting a cylindrical surface sample of a human or animal body tissue, comprising:
a tube, the inner diameter whereof determines the diameter of the sample to be collected, and exhibiting a cutting edge at its distal end,
a gripping handle, fastened to the tube in the region of a proximal end of said tube,
a knife positioned inside the tube and having a cutting edge,
a device for actuating the knife, including an actuating element connected to the gripping handle so as to give the knife a helical motion in the direction of a distal end of the tube, then a rotary motion for detaching the sample from the tissue.

2. The device of claim 1, wherein the actuating element is arranged so that, after said rotary motion and under the influence of pressure exerted toward the distal end, said actuating element causes the knife to slide inside the tube so as to make the knife protrude from the distal end of the tube for the purpose of releasing the collected sample.

3. The device of claim 1, wherein, said actuating device comprises:
a connecting element fastened to the tube,
an actuating rod fastened, at its distal end, to the knife,
a retaining element fastened to the actuating rod, connected to the connecting element by a helical connection in a proximal position of the actuating rod with respect to the tube and by a pivoting connection in a distal position of the actuating rod.

4. The device of claim 3, wherein said connecting element comprises
a tapped proximal portion cooperating with a threaded collar of the retaining element to form said helical connection and
a distal bore larger than said collar to form said pivoting connection.

5. The device of claim 3, wherein said actuating device comprises a thumbwheel fastened to the distal end of the actuating rod.

6. The device of claim 3, wherein the retaining element is held in the connecting element by a ring, said ring comprising a proximal stop for the retaining element with respect to the connecting element, said stop being retractable to release the retaining element from the connecting element.

7. The device of claim 1, wherein the inner diameter of the tube is comprised between 2 and 5 mm.

8. The device of claim 1, wherein the longitudinal travel of the knife in its helical motion is comprised between 1 and 5 mm.

9. The device of claim 1, wherein the gripping handle has the general shape of a pistol, the actuating element including a trigger.

10. The device of claim 9, wherein the gripping handle further comprises a safety button movable between a locking position and a position freeing the trigger.
